(19) United States
(12) Patent Application Publication
(10) Pub. No.: US 2024/0108303 A1
(43) Pub. Date: Apr. 4, 2024

(54) METHOD FOR CARDIAC AUSCULTATION USING BLOOD PRESSURE CUFF

(71) Applicant: California Institute of Technology, Pasadena, CA (US)

(72) Inventors: Alessio TAMBORINI, PASADENA, CA (US); Morteza GHARIB, ALTADENA, CA (US)

(73) Assignee: California Institute of Technology, Pasadena, CA (US)

(21) Appl. No.: 18/372,626

(22) Filed: Sep. 25, 2023

Related U.S. Application Data
(60) Provisional application No. 63/409,610, filed on Sep. 23, 2022.

(51) Int. Cl. A61B 7/00; A61B 5/00; A61B 5/021; A61B 5/022

(52) U.S. Cl. CPC A61B 7/00; A61B 5/02116; A61B 5/022; A61B 5/7264; A61B 5/7282; A61B 5/025

(57) ABSTRACT

The disclosure relates to systems and methods for noninvasively performing cardiac auscultation. In some implementations, a method includes: performing, using a BP cuff of a BP cuff system, a blood pressure measurement of a subject to obtain one or more blood pressure values corresponding to the subject; inflating, based on the one or more blood pressure values corresponding to the subject, the BP cuff to a subject specific pressure value; capturing, using the BP cuff system, while the BP cuff is inflated to the subject specific pressure value, a pulse pressure waveform signal associated with an artery of the subject; obtaining a sound waveform signal associated with a heart valve of the subject by filtering the pulse pressure waveform signal to extract sound components associated with the opening or closing of the heart valve; and analyzing the sound waveform signal to identify characteristics of the heart of the subject.

### CROSS-REFERENCE TO RELATED APPLICATIONS

This application claims the benefit of U.S. Provisional Patent Application No. 63/409,610, filed Sep. 23, 2022, and titled “Method For Cardiac Auscultation Using Blood Pressure Cuff”, which is incorporated herein by reference in its entirety.

### BACKGROUND

The generation of heart sounds is a complex physiological process caused by the interaction of heart valves and pressure gradients. Heart valves are flexible membranes that can stretch and recoil. During the cardiovascular cycle, the heart contracts and relaxes generating pressure rises and falls. These pressure fluctuations open and close valves, forcing blood to move through the heart and into the cardiovascular system. The ability of the heart to generate forceful contractions and relaxations is fundamental for the correct functionality of this muscular organ. Inability to generate these pressure differences leads to heart failure.

A beating heart produces brief transient sounds from the opening and closure of the valves. These sounds are unique to the shape and movement of the valve, allowing to identify each valve from the sound characteristics. During heart valve disease, modification in the structure of a valve will also change the sound it produces. Diseased valves generate murmurs and rubs that are specific to the condition affecting the valve.

The left ventricle (LV) is responsible for pumping blood to the cardiovascular system. At the start of systole, the LV undergoes a rapid pressure rise, the isovolumetric contraction, which causes the aortic valve (AV) to open forcefully. This process generates the “Aortic Ejection Sound” (AES), which relates to the forceful ejection of blood into the aorta. All else being equal, increased contractility, producing a faster pressure rise, will result in an AES of greater amplitude. The opposite is also true. At the end of systole, the LV undergoes a rapid pressure fall, the isovolumetric relaxation, which brings the AV to slam shut. This process is the origin of the second heart sound, a high-frequency pressure oscillation generated by the stretch and recoil of the flexible valve upon closing. The stretch and recoil cause opposing pressure expansions and contractions in the LV and aorta. All else being equal, a faster rate of pressure fall results in a larger valve oscillatory amplitude and, thus, larger pressure fluctuations.

Conventionally, in clinical cardiology, heart sounds are qualitatively auscultated with a stethoscope. The common practice with cardiologists is to place the stethoscope on four different chest areas to listen to heart sounds. Analyzing the intensity, pitch, duration, timing, and interval of the cardiac sounds allows to generate a diagnosis. Yet this methodology is qualitative and subjective, and it can have signal degradations from breathing and blood flow, all of which are not ideal for a diagnostic procedure. In addition, multiple valves open and close simultaneously, making it difficult for an operator to isolate a particular heart sound using the stethoscope selectively.

### SUMMARY

The technology described herein relates to techniques for noninvasively and quantitatively performing cardiovascular auscultation using a blood pressure (BP) cuff.

In one embodiment, a method for noninvasively performing cardiac auscultation, comprises: performing, using a BP cuff of a BP cuff system, a blood pressure measurement of a subject to obtain one or more blood pressure values corresponding to the subject; inflating, based on the one or more blood pressure values corresponding to the subject, the BP cuff to a subject specific pressure value; capturing, using the BP cuff system, while the BP cuff is inflated to the subject specific pressure value, a pulse pressure waveform signal associated with an artery of the subject; obtaining a sound waveform signal associated with a heart valve of the subject by filtering the pulse pressure waveform signal to extract sound components associated with the opening or closing of the heart valve; and analyzing the sound waveform signal to identify characteristics of the heart of the subject.

In some implementations, the one or more blood pressure values comprise a systolic blood pressure (SBP) of the subject; and the subject specific pressure value comprises a supra systolic blood pressure (sSBP) greater than the SBP.

In some implementations, the BP cuff is a brachial cuff, the artery is a brachial artery of the subject, and the heart valve is an aortic valve of the subject.

In some implementations, the method further comprises: prior to analyzing the sound waveform signal, indexing the sound waveform signal to identify cardiac cycle events of the subject.

In some implementations, indexing the sound waveform signal comprises indexing, based on the pulse pressure waveform signal, the sound waveform signal to identify the cardiac cycle events.

In some implementations, the method further comprises: capturing, concurrently to the BP cuff system capturing the pulse pressure waveform signal, an electrocardiogram (ECG) of the subject; and indexing the sound waveform signal comprises indexing, based on the ECG, the sound waveform signal to identify the cardiac cycle events.

In some implementations, filtering the pulse pressure waveform signal to extract sound components associated with the opening or closing of the heart valve comprises filtering the pulse pressure waveform signal to exclude signal components having a frequency below about 18 Hz.

In some implementations, filtering the pulse pressure waveform signal to extract sound components associated with the opening or closing of the heart valve further comprises filtering the pulse pressure waveform signal to exclude signal components having a frequency above about 250 Hz.

In some implementations, analyzing the sound waveform signal to identify characteristics of the heart comprises: analyzing a first component of the sound waveform signal associated with opening of the heart valve; or analyzing a second component of the sound waveform signal associated with closing of the heart valve.

In some implementations, analyzing the sound waveform signal to identify characteristics of the heart comprises measuring a stiffness of the heart valve based on an amplitude of the first component or the second component of the sound waveform signal during one or more cardiac cycles.

In some implementations, analyzing the sound waveform signal to identify characteristics of the heart comprises measuring a stiffness of the heart valve based on a presence or absence of the first component or second component of the sound waveform signal during one or more cardiac cycles.

In some implementations, analyzing the sound waveform signal to identify characteristics of the heart comprises measuring a contraction strength of the heart based on one or more parameters of the first component of the sound waveform signal associated with opening of the heart valve.

In some implementations, analyzing the sound waveform signal to identify characteristics of the heart comprises measuring a relaxation strength of the heart based on one or more parameters of the second component of the sound waveform signal associated with closing of the heart valve.

In some implementations, analyzing the sound waveform signal to identify characteristics of the heart comprises: generating, based on one or more features of the sound waveform signal, using a trained machine learning model, a prediction output indicating whether or not the sound waveform signal is associated with a heart condition.

In one embodiment, a non-transitory computer-readable medium has executable instructions stored thereon that, when executed by a processor, cause the processor to perform operations comprising: obtaining a pulse pressure waveform signal associated with an artery of a subject; obtaining a sound waveform signal associated with a heart valve of the subject by filtering the pulse pressure waveform signal to extract sound components associated with the opening or closing of the heart valve; indexing the sound waveform signal to identify cardiac cycle events of the subject; and analyzing the indexed sound waveform signal to identify characteristics of the heart of the subject.

In some implementations of the non-transitory computer-readable medium, filtering the pulse pressure waveform signal to extract sound components associated with the opening or closing of the heart valve comprises filtering the pulse pressure waveform signal to exclude signal components having a frequency below about 18 Hz.

In some implementations of the non-transitory computer-readable medium, filtering the pulse pressure waveform signal to extract sound components associated with the opening or closing of the heart valve further comprises filtering the pulse pressure waveform signal to exclude signal components having a frequency above about 250 Hz.

In some implementations of the non-transitory computer-readable medium, analyzing the indexed sound waveform for potential heart conditions comprises: analyzing a first component of the sound waveform signal associated with opening of the heart valve; or analyzing a second component of the sound waveform signal associated with closing of the heart valve.

In some implementations of the non-transitory computer-readable medium, analyzing the indexed sound waveform signal to identify characteristics of the heart comprises measuring a stiffness of the heart valve, a contraction strength of the heart, or a relaxation strength of the heart based on one or more features of the first component of the sound waveform or the second component of the sound waveform.

In some implementations of the non-transitory computer-readable medium, analyzing the indexed sound waveform signal to identify characteristics of the heart comprises: generating, based on one or more features of the sound waveform signal, using a trained machine learning model, a prediction output indicating whether or not the sound waveform signal is associated with a heart condition.

Other features and aspects of the disclosed technology will become apparent from the following detailed description, taken in conjunction with the accompanying drawings, which illustrate, by way of example, the features in accordance with implementations of the disclosed technology. The summary is not intended to limit the scope of any inventions described herein, which are defined by the claims and equivalents.

### BRIEF DESCRIPTION OF THE DRAWINGS

The present disclosure, in accordance with one or more implementations, is described in detail with reference to the following figures. The figures are provided for purposes of illustration only and merely depict example implementations. Furthermore, it should be noted that for clarity and ease of illustration, the elements in the figures have not necessarily been drawn to scale.

FIG. 1 is a high level diagram depicting a system within which the technology described herein can be implemented to provide quantitative cardiac auscultation using a BP cuff, in accordance with some implementations of the disclosure.

FIG. 2 illustrates a mode of vibration of an aortic valve cusp between the aorta and left ventricle.

FIG. 3A depicts a mode of vibration of a normal aortic valve.

FIG. 3B depicts a mode of vibration of an aortic valve having a stiffened leaflet.

FIG. 4A depicts a mode of vibration of an aortic valve performing a high strength contraction.

FIG. 4B depicts a mode of vibration of an aortic valve performing a normal strength contraction.

FIG. 4C depicts a mode of vibration of an aortic valve performing a low strength contraction.

FIG. 5 is an operational flow diagram illustrating an example method that can be implemented to provide quantitative cardiac auscultation using a BP cuff, in accordance with some implementations of the disclosure.

FIG. 6 includes plots showing a measured pulse pressure waveform, a sound waveform extracted from the pulse pressure waveform, and a measured ECG for a healthy subject.

FIG. 7 includes plots showing a measured pulse pressure waveform, a sound waveform extracted from the pulse pressure waveform, and a measured ECG for a subject having heart valvular disease.

FIG. 8A contains plots showing sound waveforms obtained for three different subjects by filtering measured pulse pressure waveforms, and plots showing corresponding ECG waveforms.

FIG. 8B includes a plot showing S1 and S2 timing data for one cardiac cycle for one of the subjects of FIG. 8A

FIG. 9A contains plots showing pressure-sound waveforms obtained for two subjects having healthy aortic valves.

FIG. 9B contains plots showing pressure-sound waveforms obtained for two subjects having aortic valve stenosis.

FIG. 10 contains plots showing a simultaneous recording of cuff pressure-sound, a left ventricle signal obtained using a catheter, and an ECG.

FIG. 11 is a diagram showing an example of a blood pressure cuff system, in accordance with some implementations of the disclosure.

FIG. 12A is a diagram showing an example of a resistive component having a fixed orifice, in accordance with some implementations of the disclosure.

FIG. 12B is a diagram showing an example of a resistive component having an in-line filter, in accordance with some implementations of the disclosure.

FIG. 12C is a diagram showing an example of a resistive component having a reduced internal diameter, in accordance with some implementations of the disclosure.

FIG. 12D is a schematic of the capacitive component including an elastic tube, in accordance with some implementations of the disclosure.

FIG. 12E is a schematic of the capacitive component including tube with a piston cylinder, in accordance with some implementations of the disclosure.

FIG. 13 is a flow diagram showing an example of a pulse pressure waveform measurement method, in accordance with some implementations of the disclosure.

FIG. 14A is an example plotting of a left ventricle pressure-volume (“PV”) loop in a young patient, in accordance with some implementations of the disclosure.

FIG. 14B is an example plotting of a left ventricle pressure-volume (“PV”) loop in an old patient, in accordance with some implementations of the disclosure.

FIG. 15 is a flow diagram showing an example of a left ventricular end-diastolic pressure risk prediction method, in accordance with some implementations of the disclosure.

FIG. 16 is an example plotting of an envelope function reconstructed at a specified pressure level and three pressure holds, in accordance with some implementations of the disclosure.

FIG. 17 is an example plotting of an envelope function to estimate SBP and DBP changes following pulse amplitude fluctuations, in accordance with some implementations of the disclosure.

The figures are not exhaustive and do not limit the present disclosure to the precise form disclosed.

### DETAILED DESCRIPTION

The current gold standard for cardiac auscultation, a stethoscope, relies on a qualitative and subjective assessment by a cardiologist listening to heart sounds in multiple chest areas. During this process, the cardiologist attempts to position the stethoscope over different heart valves and listen to the opening and closing of these valves, with the closure of the valves being the sound that is primarily heard. When heart valves disease and/or age, the mechanism with which they close changes, affecting the sounds that they produce. Although this subjective assessment can be used to provide an initial diagnosis indicating that something is potentially wrong with the heart valve mechanism, the diagnosis typically needs to followed up by more invasive and/or expensive tests. For example, if a cardiologist hears “snapping” or “murmur” sounds indicative of a potential condition such as stenosis, the patient may be referred for ultrasonic imaging of the heart valves. As another example, a surgical procedure that places a catheter in the heart (e.g., left heart catheterization) is commonly used to detect conditions such as a change in heart contractility. As such, there is a large medical need to develop systems to quantitatively assess heart conditions non-invasively.

To this end, the technology describe herein is directed to methods and systems for performing cardiovascular auscultation using a BP cuff assembly. By implementing the methodology described herein, heart sounds can be quantitatively measured using pulse pressure waveforms captured using the BP cuff system. In accordance with implementations, further described below, the pulse pressure waveform captured with the BP cuff system can be filtered to extract a sound waveform. The extracted sound waveform can include heart sound components associated with heart and valvular function, and it can be quantitatively analyzed to identify potential heart conditions. For example, heart conditions associated with valve stiffness and/or contraction strength, which affect heart sounds, could be quantitatively assessed based on components of the sound waveform associated with opening and/or closing of the valve.

Various advantages can be realized by implementing the technology described herein. The techniques described herein can use a single measurement modality—a blood pressure cuff assembly in combination with digital (or analog) signal processing—to extract heart sound data for quantitative assessment. This can improve the efficiency with which some cardiac conditions are diagnosed, enabling data driven diagnosis without the requirement of additional modalities (e.g., ultrasound imaging or catheterization) following a stethoscope examination. The technology described herein could be implemented to enable diagnosis in an outpatient or even in an at-home setting. These and other advantages that can be realized by implementing the technology described herein are further exemplified by the description that follows.

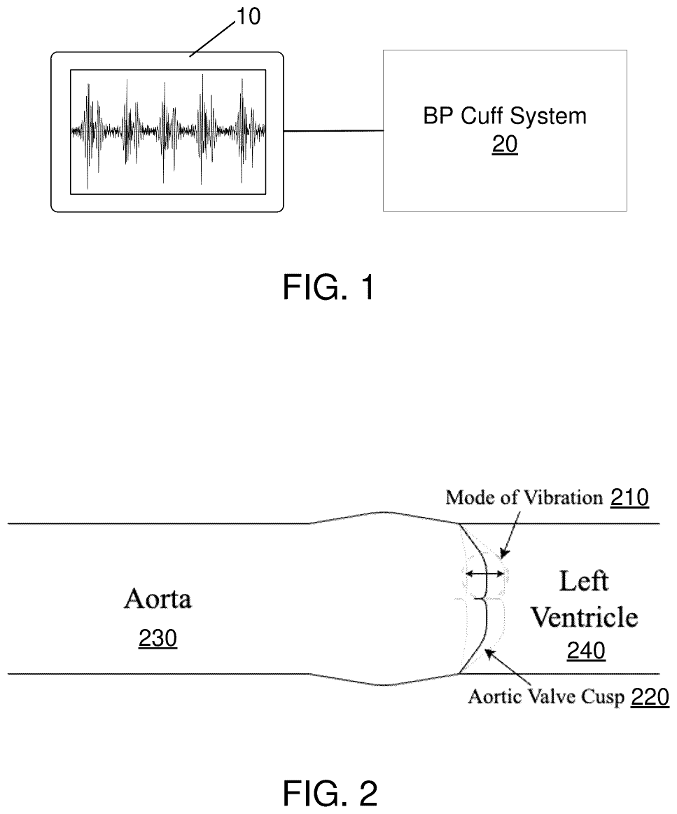

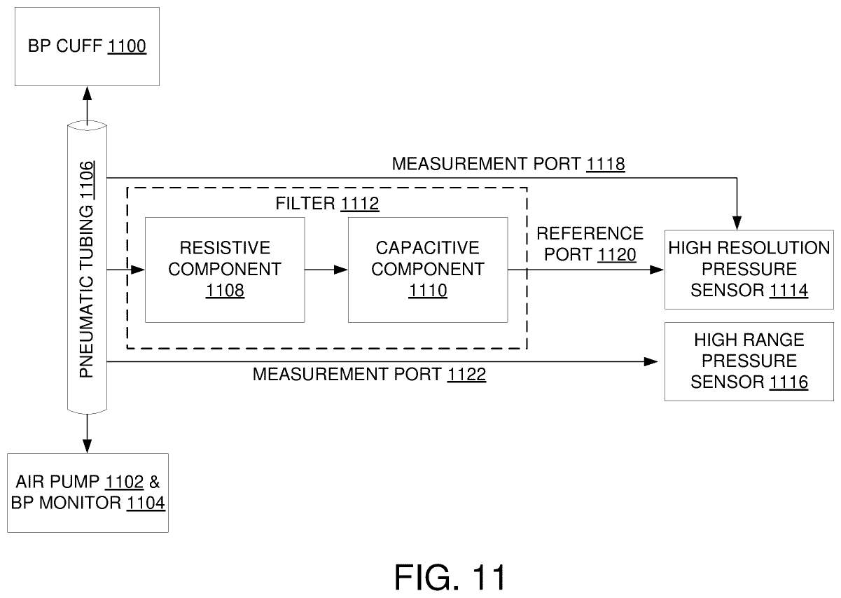

FIG. 1 is a high level diagram depicting a system within which the technology described herein can be implemented to provide quantitative cardiac auscultation. As depicted, the system includes a cardiac auscultation device 10 in communication with a BP cuff system 20. During operation, the BP cuff system 20 is configured to capture a pulse pressure waveform signal representing the high-frequency vibrations that propagate as pressure waves within the arterial system when one or more heart valves open and close. The BP cuff system can include a pneumatic assembly in combination with a BP monitor/cuff to capture the pulse pressure waveform in high resolution, enabling measurements of smaller pressure changes that some conventional systems. Particular implementations of a BP cuff system 20 that can be used to capture a pulse pressure waveform signal in enhanced resolution are further described below with reference to FIGS. 11-17. This pulse pressure waveform signal can be stored in the digital domain as pulse pressure waveform data at BP cuff system 20 and/or cardiac auscultation device 10. In some implementations, the pulse pressure waveform data can be communicated from BP cuff system 20 to cardiac auscultation device 10 via a wired or a wireless communication link. For example, a radio frequency link such as a Bluetooth® link or a Wi-Fi® link can be used to communicate the data.

The cardiac auscultation device 10 is configured to process the pulse pressure waveform signal to extract heart sound data. The heart sound data can include components corresponding to the opening and closing of a heart valve such as the aortic valve. The heart sound data can be stored at cardiac auscultation device 10. The cardiac auscultation device 10 can further process the heart sound data to predict whether or not the associated patient potentially suffers from a cardiac condition. For example, the data can indicate whether or not an aortic value is cyclically opening and closing as expected. The particular methods that can be implemented by cardiac auscultation device 10 to quantitatively perform cardiac auscultation and/or diagnosis cardiac conditions based on the heart sound data are further described below.

In this particular example, cardiac auscultation device 10 is illustrated as a mobile device in communication with BP cuff system 20. The mobile device can be a smartphone, a tablet, laptop, a smartwatch, a head mounted display (HMD), or other suitable mobile device configured to generate the heart sound data. In a particular implementation, the mobile device can run an application for performing cardiac auscultation. The application can be configured to display the pulse pressure waveform data and/or the derived heart sound data. The application can also be configured to display an expected diagnosis based on processing the heart sound data. In other implementations, a desktop computer or other device can be implemented as cardiac auscultation device 10. In yet other implementations, cardiac auscultation device 10 and BP cuff system 20 can be integrated as part of the same system. For example, the functions and/or hardware of cardiac auscultation device 10 can be incorporated into BP cuff system 20. In some implementations, cardiac auscultation device 10 can be a device specifically dedicated to performing cardiac auscultation. For example, the device 10 can be designed with an integrated circuit that specifically performs the cardiac auscultation functions described herein. In such implementations, signal processing could take place in the analog domain.

To understand the technology described herein, it is instructive to first consider the mechanism of heart sound generation by valve movements, which enables quantitative measurement of the heart sounds that can be used for diagnosing cardiac conditions. Heart sounds are high-frequency pressure vibrations generated from valve motion that propagate along the arterial system as pressure waves. For example, FIG. 2 illustrates a mode of vibration 210 of the aortic valve cusp (leaflet) 220 between the aorta 230 and left ventricle 240. In this example, the aortic valve leaflet 220 opens and closes based on a pressure difference between the aorta 230 and left ventricle 240. Upon closing, the valve leaflets oscillate up and down.

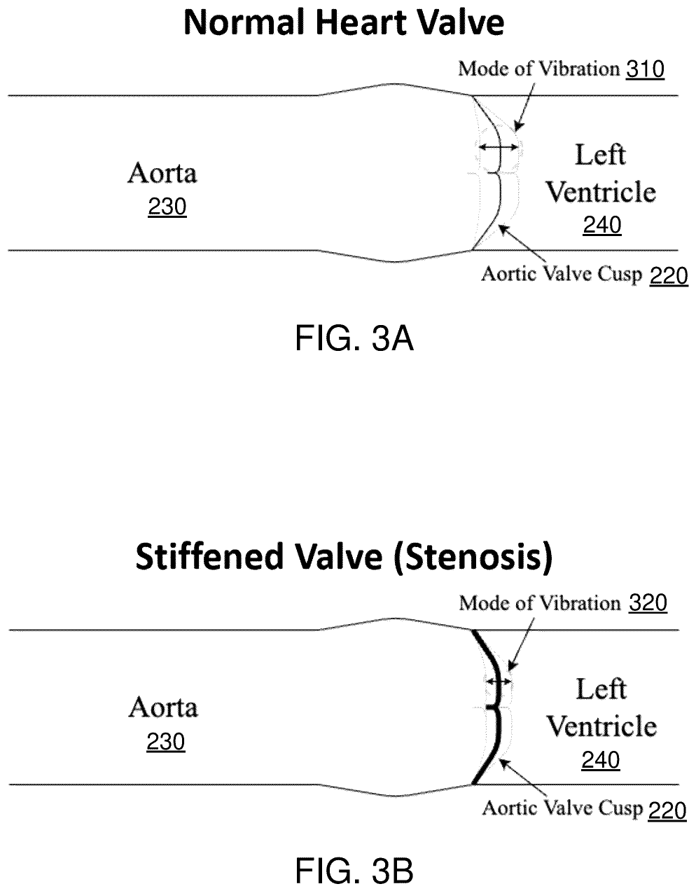

One factor that affects heart sounds is valve stiffness, which affects the mechanics of the valve. For example, FIGS. 3A-3B depict the alteration of heart sounds caused by valve leaflet stiffening, i.e., a medical condition referred to as stenosis. FIG. 3A depicts a normal aortic valve with a leaflet mode of vibration 310. The normal aortic valve has a flexible leaflet that oscillates with a given amplitude. FIG. 3B depicts an aortic valve with a stiffened leaflet (depicted by dark shaded line) with a mode of vibration 320. As depicted, the stiffened leaflet oscillates with a much lower amplitude of vibration. As such, as compared to a normal valve, it can be expected for a stenotic valve that the motion (e.g., stretch and recoil) of the valve will be impaired, and this will modify the pressure vibrations that are generated.

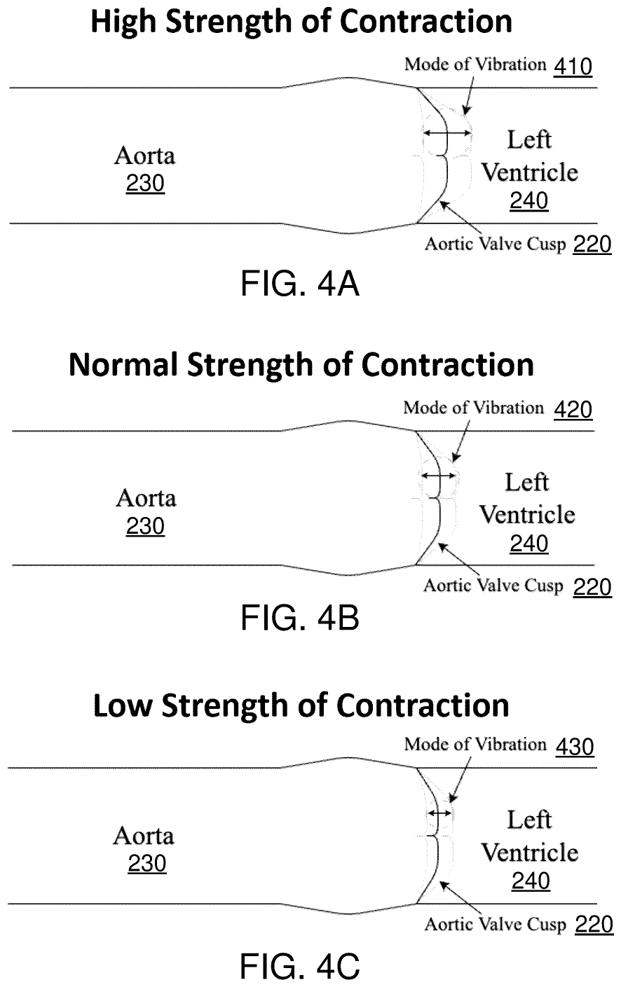

Another factor that affects heart sounds is contraction strength, which is the force that is used to open and close a valve. For example, FIGS. 4A-4C depict the alteration of heart sounds caused by different heart contraction rates. FIG. 4A depicts the aortic valve performing a high strength of contraction having a mode of vibration 410. FIG. 4B depicts the aortic valve performing a normal strength of contraction having a mode of vibration 420. FIG. 4C depicts the aortic valve performing a low strength of contraction having a mode of vibration 430. Heart contraction and relaxation strength are proportional to the rate of pressure change across the valve. A well-functioning heart will typically generate stronger contractions. As an individual ages, the heart typically loses contraction strength. As depicted by FIGS. 4A-4C, a stronger contraction will generate larger valve stretch/recoil. This in turn generates larger amplitude pressure vibrations.

The aforementioned pressure vibrations propagate as pressure waves within the arterial system. It has been discovered by the inventors that using a modified BP cuff system, these pressure vibrations can be measured noninvasively by capturing a pulse pressure waveform. Particular BP cuff systems for making such quantitative measurements are further described below with reference to FIGS. 11-17, but it should be appreciated that the techniques described herein for performing cardiovascular auscultation need not be limited to such systems, and they can be performed in conjunction with any BP cuff system that can capture pulse pressure waveform data in sufficiently high resolution.

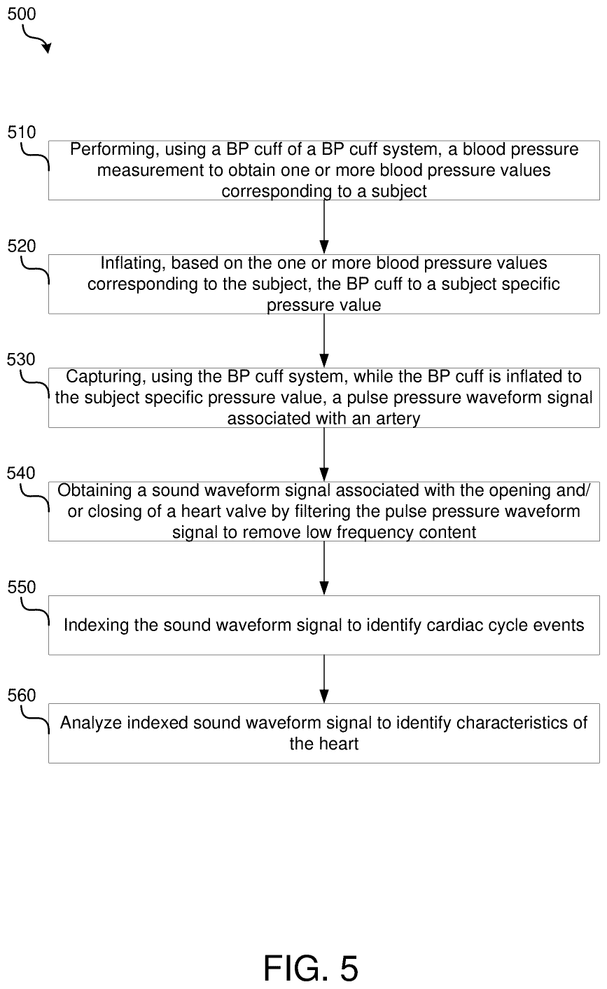

FIG. 5 is an operational flow diagram illustrating an example method 500 that can be implemented to provide quantitative cardiac auscultation using a BP cuff, in accordance with some implementations of the disclosure. In some implementations, one or more of the operations of method 500 can be performed by cardiac auscultation device 10 and/or BP cuff system 20 in response to executing, using a processing device, instructions stored thereon. In some implementations, method 500 can be implemented as two independent methods by BP cuff system 20 and cardiac auscultation device 10. For example, in some implementations operations 510-530 can correspond to a method that is performed by a BP cuff system to capture a pulse pressure waveform signal, and operations 540-560 can correspond to a subsequent method of cardiac auscultation that can be performed by a cardiac auscultation device 10 using the pulse pressure waveform signal. In some implementations cardiac auscultation device 10 can perform operations 540-560 in response to executing instructions corresponding to an application for performing quantitative cardiac auscultation

Operation 510 includes performing, using a BP cuff of a BP cuff system, a blood pressure measurement to obtain one or more blood pressure values corresponding to a subject. For example, the blood pressure measurement can be performed using a brachial cuff that is placed on the subject's left or right arm. The blood pressure measurement can provide subject specific blood pressure values such as a SBP and/or a diastolic blood pressure (DBP). For example if the subject has a normal blood pressure, the subject specific blood pressure values can range between 90/60 millimeters of mercury (mmHg) and 120/80 mmHg. These blood pressure values can be used as a baseline when performing subsequent operations of method 500. Specifically, the blood pressure values can provide a guideline of how the force that is applied from the cuff to the artery (e.g., brachial artery) will change the measured signal.

Operation 520 includes inflating, based on the one or more blood pressure values corresponding to the subject, the BP cuff to a subject specific pressure value. Operation 530 includes, capturing, using the BP cuff system, while the BP cuff is inflated to the subject specific pressure value, a pulse pressure waveform signal associated with an artery.

For example, to measure the sound waves at a brachial artery, an arm cuff system that measures a pulse waveform with high resolution can be used. Such a design can be configured to use a blood pressure monitor with tourniquet capabilities to inflate and hold a specific pressure in the cuff. During the hold pressure period, a high-resolution pressure sensor can used to measure the pulse pressure waveform with fast enough sampling to record heart sounds.

As discussed above, cardiac auscultation performed at the brachial artery using a brachial cuff is a pressure phenomenon; the pressure vibrations generated by the valve opening/closing travel as pressure waves along the arterial system. The systems and methods described herein are capable of capturing pressure waves in high resolution, and they are therefore capable of resolving these oscillations. The stethoscope, on the other hand, uses its flexible diaphragm to capture sound waves propagating through soft tissue. Given the signal is embedded in the pressure signal propagating along the artery, application of a stethoscope at arm on the brachial artery location will not recover the pressure pulsation. As such, one advantage of implementing the technology described herein is that a stethoscope system cannot be used as a substitute for this pressure-based measurement.

In various implementations, the subject specific pressure value is selected to remove other sources of noise besides the sounds created by the opening and closing of the valve. In particular, the subject specific pressure value can be selected to reduce or remove any noise created by local blood flow vibrations. To this end, in some implementations the subject specific pressure value can be selected to be greater than a SBP measured at operation 510. This pressure above SBP can be referred to as a sSBP. The sSBP can be set to some threshold value above the measured SBP that ensures the artery is fully constricted. In a particular embodiment, the sSBP can be set to about SBP+35 mmHg.

When an internal pressure is present in the artery and an external pressure is applied that is greater than this internal pressure, the artery can be fully closed, removing any noise created by local blood flow vibrations. This can be important as flow in an elastic artery can cause flutter-like noise in the same frequency range as heart sounds, thereby obscuring the sound from the heart valve closure. As such, a fully obstructive/occlusive pressure (cuff pressure greater than blood pressure) can be used to generate a signal that is free from local flow vibrations. By virtue of setting the subject specific pressure value to remove local noise, a cleaner measurement of the opening and closing of a heart valve can be obtained.

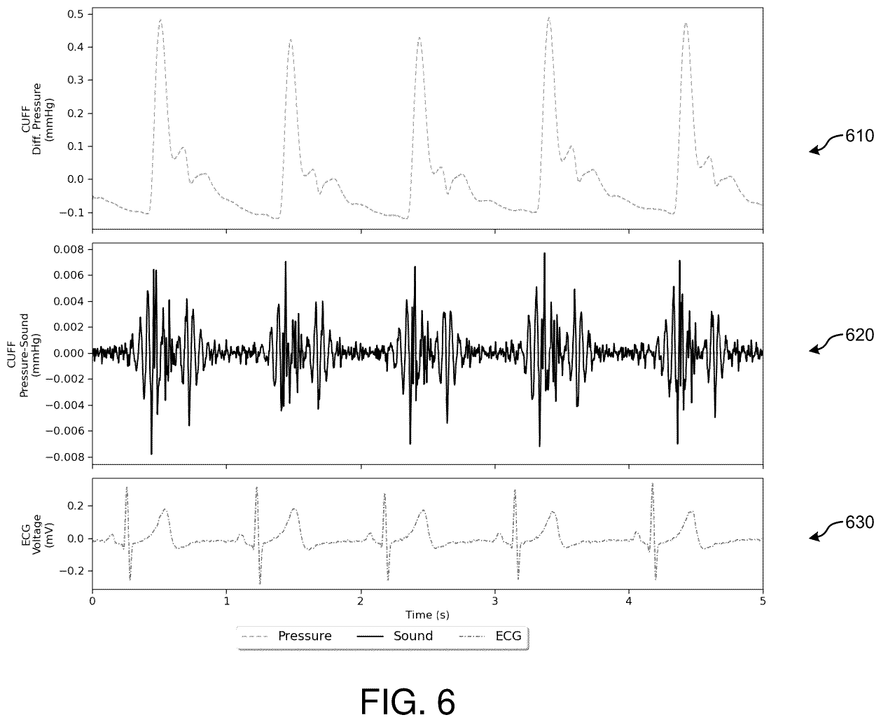

By way of illustration, plot 610 of FIG. 6 shows pulse pressure waveform data that was measured for a healthy subject using the sSBP hold pressure on the left brachial artery. Plot 630 shows an ECG captured for the subject at the same time as a reference.

In alternative implementations, the subject specific pressure value can be selected to be below sSBP. For example, the pressure-sound measurement can be performed at physiological hold pressures such as, but not limited to, sub-DBP, DBP, mean arterial pressure (MAP), or SBP. As noted above, the hold pressure affects the pressure and flows at the measurement location. While the sSBP hold may be the preferred condition to extract heart sounds directly, signal processing methods can be used to isolate heart sounds from flow-related sounds in the hold pressures with the flow in the arm.

In alternative implementations, it may be desirable to extract the local flow vibration sounds (e.g., Korotkoff sounds) generated in the artery. In such implementations, the subject specific pressure value can be set to be below sSBP. In such alternative implementations, which are outside the scope of method 500, the aortic or other heart sounds can be filtered out.

In some implementations, the pulse pressure waveform signal can be captured in the analog domain. In some implementations, the pulse pressure waveform signal can be captured as pulse pressure waveform data in the digital domain.

Particular implementations of systems and methods for non-invasive pulse pressure waveform measurement are further described below with reference to FIGS. 11-17

Operation 540 includes obtaining a sound waveform signal (also referred to herein as a pressure-sound waveform signal) associated with the opening and/or closing of a heart valve by filtering the pulse pressure waveform signal to remove low frequency content. Similar to pulse pressure waves, sound waves propagating along the cardiovascular system will cause measurable temporal pressure signature. The amplitude of this sound signal will be much smaller than the pulse pressure waveform signal. The sound signal can be resolved by filtering the pulse pressure waveform signal to isolate the sound elements within the recording.

The pressure waveform signal can be filtered to remove oscillatory components having a frequency below a certain frequency threshold. The threshold can be set to about 18 Hz as the majority of heart sounds can be expected to occur at about 18 Hz or higher. As such, certain “slow” oscillatory movements associated with the pulse pressure waveform signal can be filtered out to extract the sound signal. In some implementations, high-pass and/or band-pass filters can be employed to remove the components below the threshold frequency.

In some implementations, the pressure waveform signal can also be filtered to remove oscillatory components having a frequency above which heart sounds are expected to occur. As such, the pressure waveform signal can be filtered to remove oscillatory components having a frequency outside a range in which heart sounds are expected to occur. For example, as the majority of heart sounds can be expected to occur in about the 18 to 250 Hz range, in some implementations one or more filters may be applied to the pulse pressure waveform signal to exclude signal components having a frequency below about the 18 Hz range and also to exclude signal components having a frequency above about the 250 Hz range. As such, in some implementations any suitable filter such as a band-pass filter, a low-pass filter, a high-pass filter, or some combination thereof can be used to filter the pulse pressure waveform signal. In some implementations, filtering can occur over multiple frequency ranges of interest, where each frequency range may be associated with a type of heart sound of interest.

In some implementations, a narrower filtering range (i.e., narrower than 18 to 250 Hz range) can be set to identify specific types of heart sounds. For example, in some implementations a first heart sound can be detected in the range of about 18-26 Hz (i.e., a “phase one” heart sound), and a second heart sound (i.e., “phase four” heart sound) can be detected in the range of about 40-60 Hz.

By way of illustration, plot 620 of FIG. 6 shows the sound waveform extracted from the pulse pressure waveform of plot 610. As illustrated by example plots 610-620 of FIG. 6, the extracted sound waveform can have a significantly smaller amplitude (i.e., more than one order of magnitude smaller) than the pulse pressure waveform. As such, it should be appreciated that while on the scale of plot 610 the sound waveform component may not necessarily be visually perceptible to a human, it can be extracted by implementing the signal processing techniques described herein (e.g., low frequency content removal) on a sufficiently high resolution pulse pressure waveform signal.

The filtering of the pulse pressure waveform signal can be applied with hardware and/or software methods, and with digital and/or analog filters. In implementations where digital filtering is applied, the digital filtering can be applied at the time of measurement or later during processing. In such implementations, operations 540-560 can be performed as a separate method at a later time. For example, the method could be performed at a later time using a cardiac auscultation device 10 that receives a pulse pressure waveform dataset.

Operation 550 includes indexing the filtered signal into cardiac cycle events. During indexing, the signal can be segmented into physiologically significant cardiac cycle events, each segment being indexed into a time interval (e.g., a start time and end time). In some implementations, each segment can include a waveform associated with the opening and closing of the heart valve. As such, in some implementations of indexing, each cardiac cycle corresponding to the opening and closing of the heart valve (e.g., aortic valve) can be isolated as a segment. It should be appreciated that depending on the heart condition of interest, the cardiac cycle events that the signal is indexed to can vary. As such, the start and/or end times of each segment of the indexed signal can vary depending on the application. In some implementations, the start time may be associated with the opening or closing of a heart valve. In some implementations, the end time may be associated with the opening or closing of a heart valve. In some implementations, the start or end time may be associated with some other cardiac cycle event besides the opening or closing of the heart valve.

In some implementations, the previously captured pulse pressure waveform can be used to demarcate the cardiac cycle events. For instance, in the example of FIG. 6, the pulse pressure waveform associated with plot 610 can be used to perform indexing. As another example, if the measurement is taken concurrently with an ECG, the ECG can be used as the demarcation of the cardiac cycle. For instance, in the example of FIG. 6, the ECG waveform associated with plot 630 can be used to perform indexing.

In some implementations, indexing operation 550 can be skipped, and the sound waveform signal can be analyzed as a whole at operation 560.

Operation 560 includes analyzing the indexed sound waveform signal to identify characteristics of the heart. For example, a relative and/or absolute analysis of one or more of the following features of one or more segments of the sound waveform can be analyzed to identify potential heart conditions: amplitudes, timings, and frequencies. In some cases, a rate of change/derivative of the amplitude may be considered. In some implementations the analysis can be performed for a series of captured cardiac cycle events (e.g., indexed series of events). These features may be analyzed for each cardiac cycle event, independent of, or in conjunction with other cardiac cycle events.

The sound waveform can be analyzed with a variety of methods that extract quantitative parameters from an oscillatory signal, including time-domain analysis, frequency-domain analysis, and/or time-frequency-analysis. The time-domain analysis could extract parameters such as amplitude, slopes, timings, instantaneous frequency from zero crossings, and signal envelope amplitudes. The frequency-domain analysis could extract frequency composition of the signal, its amplitude distribution and the power distribution of each frequency component. The time-frequency analysis could identify the frequency content variation over time as well as using a wavelet transform identify different frequency scales of the signal.

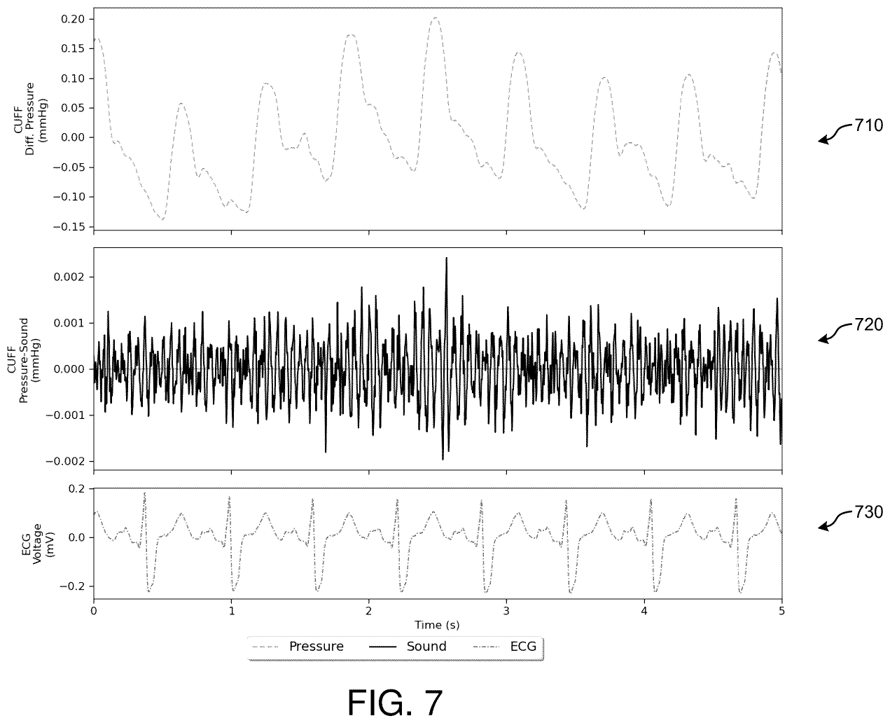

By way of illustration, FIG. 7 shows the pulse waveform measurement results for an individual with heart valvular disease. In this example, the subject has both severe mitral stenosis and regurgitation and moderate aortic stenosis. Plot 710 shows a pulse pressure waveform data that was measured for the subject using the sSBP hold pressure on the left brachial artery. Plot 720 shows the sound waveform data extracted from the pulse pressure waveform. In contrast to the sound waveform associated with the healthy subject of FIG. 6, no clear signal is present in the sound waveform associated with the subject of FIG. 7, indicating the lack of correct mitral and aortic valve function. Plot 730 shows an ECG captured for the subject at the same time as a reference.

In some implementations, one or more machine learning models can be used to classify and detect specific sound signal features and patterns for heart condition identification. A machine learning model can be trained to output, given an input sound waveform signal, a prediction indicating whether or not the waveform signal is associated with a heart condition. For example, the machine learning model can classify the sound waveform signal as being associated with normal heart function or compromised heart function. In some implementations, the model can be trained to specifically identify/classify heart conditions such as stenosis, left ventricular contraction weakening, or other heart conditions. To this end, the machine learning model can be trained using an existing dataset of sound waveform signals associated with patients having healthy hearts and patients having diseased hearts. The dataset can be manually, semiautomatically, or automatically labeled in advance of training. The model can be trained based on one or more of the aforementioned features of the waveform, including amplitudes, timings, and/or frequencies.

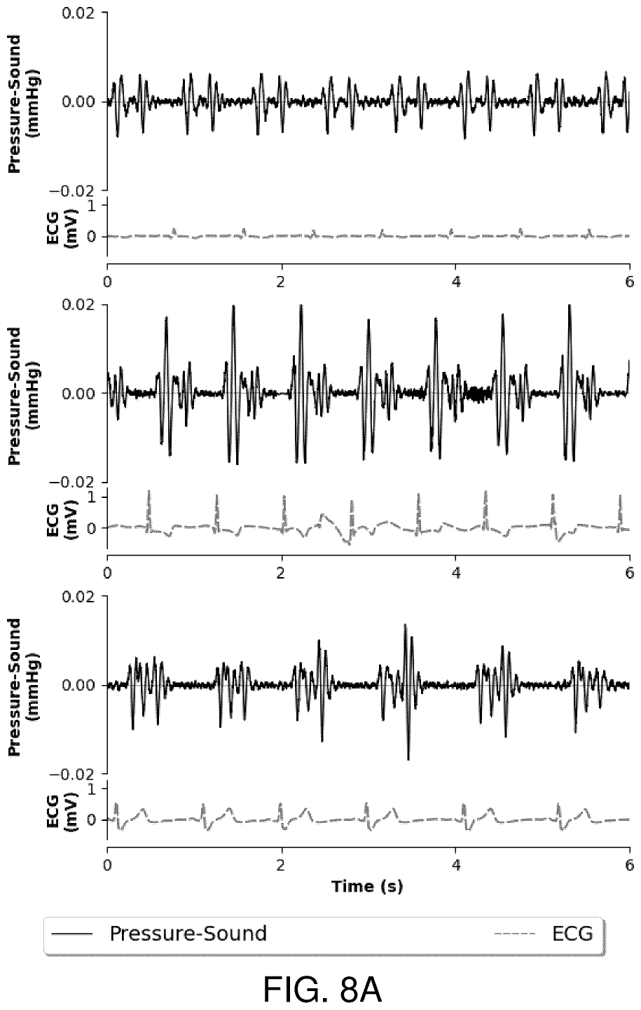

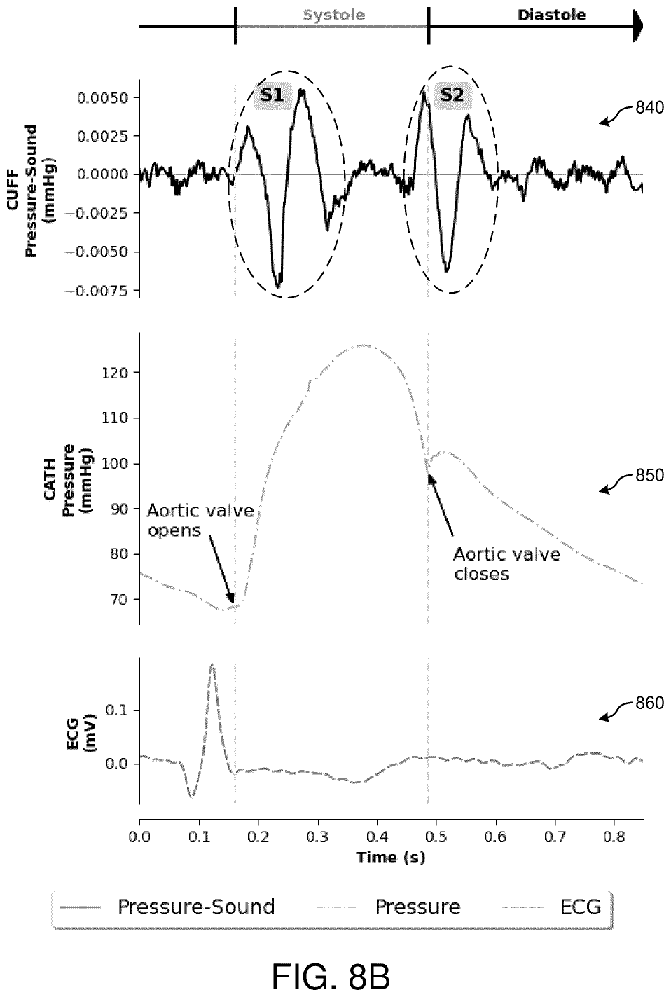

In some implementations, each cardiac cycle associated with the sound waveform can contain data corresponding to the opening (“S1” heart sound) and closing (“S2” heart sound) of a heart valve. To illustrate, FIGS. 8A-8B contain plots showing three examples of extracted sound waveforms for three different subjects, and the relative timing with the pressure waveform, illustrating the opening and closing of the aortic valve. The illustrated data was captured using the sSBP hold pressure on the left brachial artery, and by filtering the pulse pressure waveform as described above. FIG. 8A shows the sound waveforms for the three subjects and corresponding ECG waveforms that were captured. FIG. 8B shows the S1 and S2 timing data for one cardiac cycle for the first subject of FIG. 8A. As illustrated by pressure-sound plot 840 of FIG. 8B, the sound waveform contains a first oscillation (S1) followed by a very brief transient, and a second oscillation (S2). For reference, FIG. 8B also includes a pressure plot 850 captured using a catheter, and an ECG plot 860.

### Valve Leaflet Stiffening Identification from Sound Waveform Signal

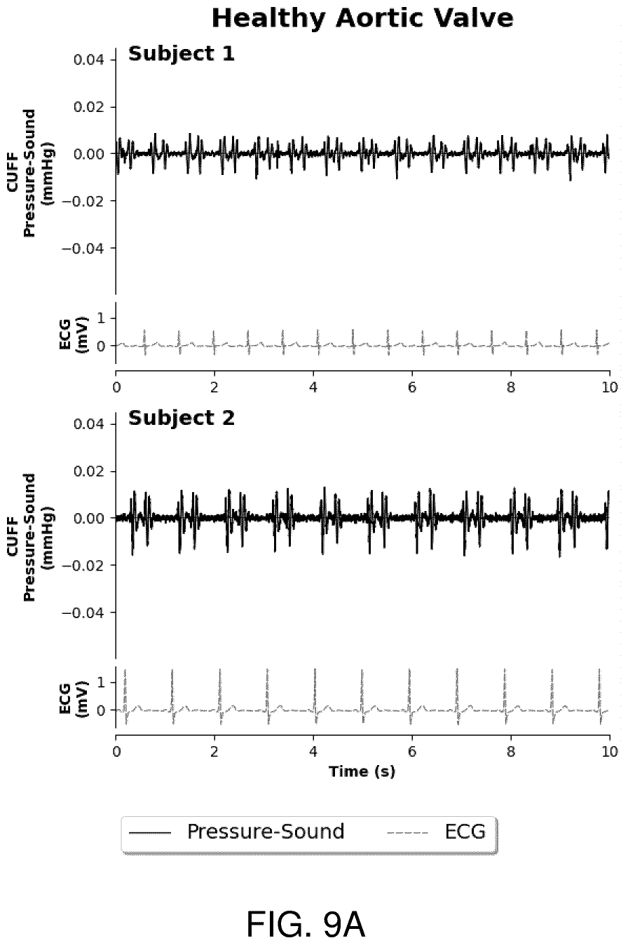

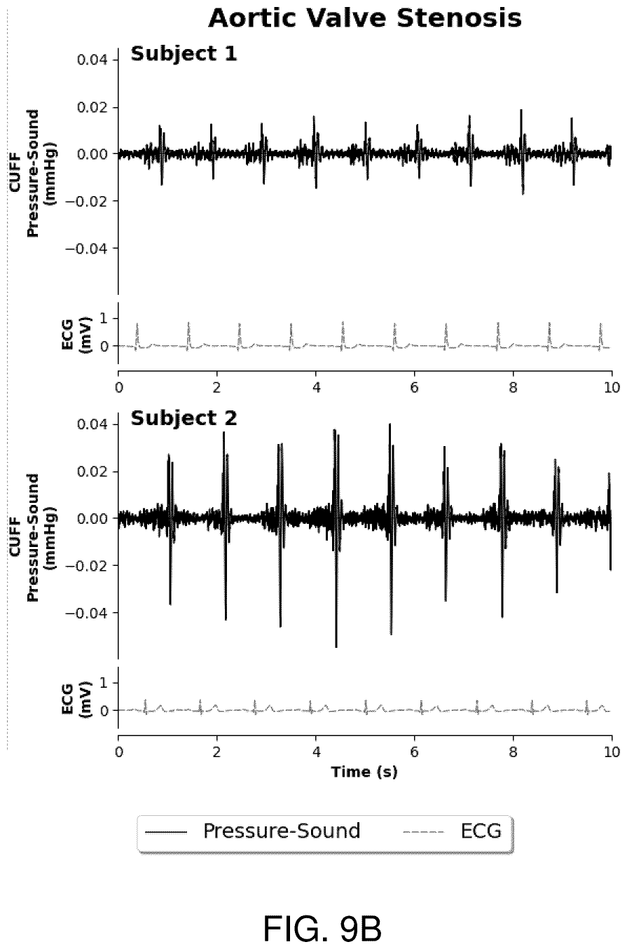

Stiffening of the aortic valve will result in impaired valve motion. This impaired valve motion can cause a reduction or complete loss of S1 and/or S2 sounds described above. Accordingly, in some implementations the sound waveform signal can be analyzed for the presence or absence of S1 and/or S2 sounds by measuring how the amplitude of the signal changes as a function of time during each cardiac cycle. To illustrate, FIGS. 9A-9B show pressure-sound waveforms measured for four different subjects. FIG. 9A shows the pressure-sound waveforms for two subjects having healthy aortic valves. In both subjects, the first and second oscillations associated with S1 and S2 sounds periodically occur as expected. FIG. 9B shows the pressure-sound waveforms for two subjects having aortic valve stenosis. In the first subject, the oscillation associated with the S1 sound is reduced. In the second subject, the oscillation associated with the S1 sound is absent. As such, in this example the opening of the valve can be quantitatively assessed to be impaired from the sound waveform signal. In other instances, it may be measured that the S2 portion of the signal is weakened or absent, and thus the closing of the valve is impaired. In yet other instances, it may be measured that both the S1 and S2 portions of the signal are weakened or absent.

### Assessment of Left Ventricular Contractility from Sound Waveform Signal

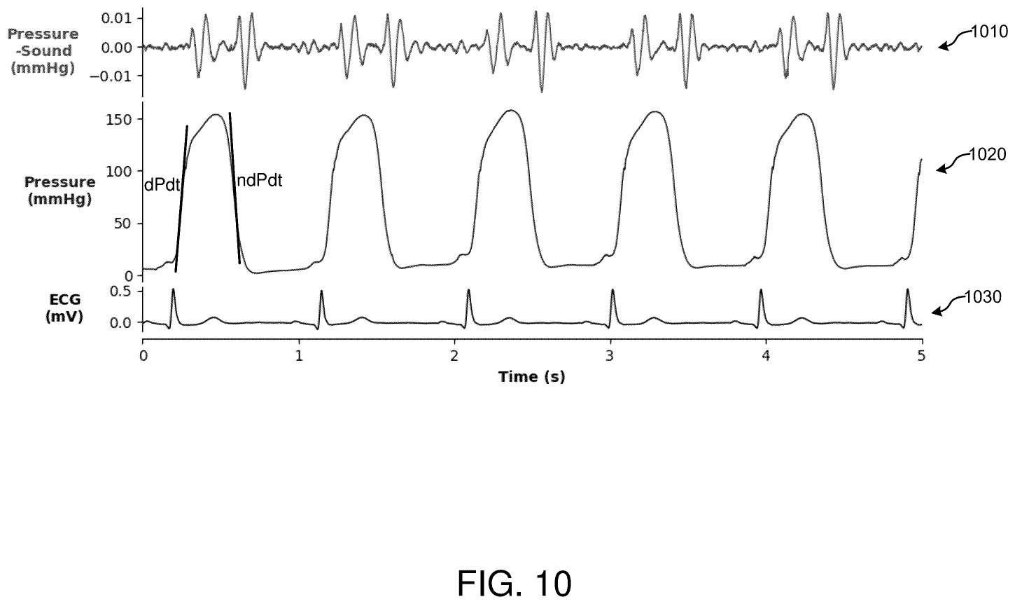

In some implementations, the LV contractile and/or relaxation rate can be quantitatively assessed by analyzing quantitative parameters extracted from the S1 and/or S2 components of the pressure-sound waveform extracted from a high-resolution BP cuff system. To illustrate the principle of assessment, FIG. 10 contains plots showing a simultaneous recording of cuff pressure-sound (plot 1010), an LV signal obtained using a catheter (plot 1020), and an ECG (plot 1030). As depicted by plot 1020, during contraction, pressure within the heart generates rapidly. During relaxation, the pressure drops quickly, and the cycle subsequently repeats. These two processes are referred as isovolumetric contraction and isovolumetric relaxation, respectively. The maximal pressure gradient in the isovolumetric contraction is depicted as dPdt, which provides a measure of LV contraction strength. The maximal negative pressure gradient in the isovolumetric relaxation is depicted as ndPdt, which provides a measure of LV relaxation strength.

During operation of the heart, the isovolumetric contraction will cause the aortic valve to open, and the isovolumetric relaxation will cause the aortic valve to close. As previously described, this opening of the valve is associated with an S1 sound, and this closing of the valve is associated with an S2 sound. Through experimentation, it was observed by the inventors that the LV dPdt parameter strongly correlated to the S1 sound parameters of the sound waveform, and the LV ndPdt parameter strongly correlated to the S2 sound parameters of the sound waveform. For example, correlations were measured between dPdt and the peak-to-peak amplitude and envelope of the S1 component of the sound waveform. Correlations were also measured between ndPdt and each of the peak-to-peak amplitude and envelope of the S2 component of the sound waveform. More generally, contraction strength and/or relaxation strength of the heart could be estimated by extracting one or more quantitative parameters from the S1 and/or S2 components of the sound waveform, using one or more of time-domain analysis, frequency-domain analysis, and/or time-frequency analysis as described above. Such quantitative parameters may include amplitudes, slopes, timings, instantaneous frequency from zero crossings, signal envelope amplitudes, amplitude distribution and power distribution of frequency components, frequency content variation over time, or other quantitative parameters. By virtue of being able to analyze the S1 and S2 components of the sound waveform signal to assess left ventricular contractility, a non-invasive approach to measuring heart function can be provided as an alternative to catheterization.

### BP Cuff System

Embodiments of the systems and methods described herein can utilize a BP cuff system including a modified BP cuff to perform non-invasive yet accurate cardiac measurements. The BP cuff system can be leveraged to perform pulse pressure waveform measurements as described herein. The BP cuff system can also perform left ventricular end-diastolic pressure (“LVEDP”) measurements, pressure-volume (“PV”) loop measurements, and other important cardiac measurements. The BP cuff system can be used in conjunction with methods for performing cardiac auscultation as described herein.

### Non-Invasive Pulse Pressure Waveform Measurement

The modified BP cuff system may be used to measure a pulse pressure waveform. In some implementations, the modified BP cuff system may include a dynamic pressure sensor instead of and/or in addition to a static pressure sensor. A high resolution pressure sensor may be included for high sensitivity signal acquisition at a specified pressure level. The high resolution pressure sensor may comprise a differential pressure sensor having a measurement port and a reference port, wherein the pressure sensor measures the difference between its measurement port and reference port. A high range absolute pressure sensor may be used to calibrate the signal. An air valve or filter may be included to maintain a specific pressure level at the reference port. Maintaining the pressure level may allow the high resolution pressure sensor to operate within its normal range. During measurement the pressure sensors may simultaneously acquire signals.

The high range pressure sensor may measure with respect to atmospheric pressure while the high resolution pressure sensor may measure with respect to a variable reference pressure. The pressure sensors may be connected in parallel to the BP cuff system. In some implementations, the high range and high resolution pressure sensors may have an operating range on the order of magnitude of the measurement and signal, respectively.

No control system may be employed in the implementations described herein. Although such control systems could be used to dynamically control an air valve to open and close the valve at appropriate pressures to ensure the signal is captured correctly, in such a system, pressure fluctuations may cause sensor saturation resulting in a critical fault, signal drift, and loss of valuable information. For these reasons, an air valve system may present drawbacks that are not present in the implementations described herein.

In some implementations, a passive and self-adjusting non-invasive pulse pressure waveform measurement system may include high resolution pressure sensors in an inflatable pressurized air chamber having resistive and capacitive components. Hydraulic filtering may be implemented through a geometrical condition to passively generate a signal that only transits desired frequencies. In some embodiments an air valve may be replaced with a hydrodynamic filter. A hydrodynamic filter may comprise a fixed or adjustable orifice, an in-line filter, and/or tubing with an internal diameter (“ID”) significantly smaller than the ID of the rest of tubing included in a modified BP cuff system.

The hydrodynamic filter can be achieved using a sequential combination of resistive tubing and compliant tubing. The resistive tubing generates a flow resistance limiting the flow that can move across this element. The compliant tubing stores injected volume at a desired compliance rate. This hydraulic system generates the electrical equivalent of an RC low pass filter. In such a hydraulic system, the circuit may be understood as the time the compliant element needs to fill up through the resistive element.

In some implementations, the hydrodynamic filter comprises a resistive component and a compliant component. The resistive component is configured to impose a resistance to flow, thereby slowing down the flow, whereas the compliant component comprises a capacitive component configured to reduce pressure changes by storing air volume. In such embodiments, the compliant component may comprise tubing that connects the resistive component to the reference port. Additionally, the resistive component of the hydrodynamic filter may comprise rigid tubing with an internal diameter in the range of 10-200 μm. In some cases, an elasticity of the capacitive element is in the range of 0.2-2.0 MPa. The hydrodynamic filter may form an input connection to the reference port and thus regulate flow into the reference port. This configuration may provide the BP cuff system with steady pressure resulting in a smooth signal.

FIG. 11 shows an example of a BP cuff system having a passive configuration for the high accuracy sensor. As shown in FIG. 11, the system may include a BP cuff 1100. The system may also include pneumatic connections 106 for a high resolution pressure sensor 1114, a filter 1112 including a resistive component 1108 in series with a capacitive component 1110, and a reference port 1120. The system may also include an air pump 1102 and a BP monitor 1104. The air pump 1102 may be used to inflate the cuff 1100.

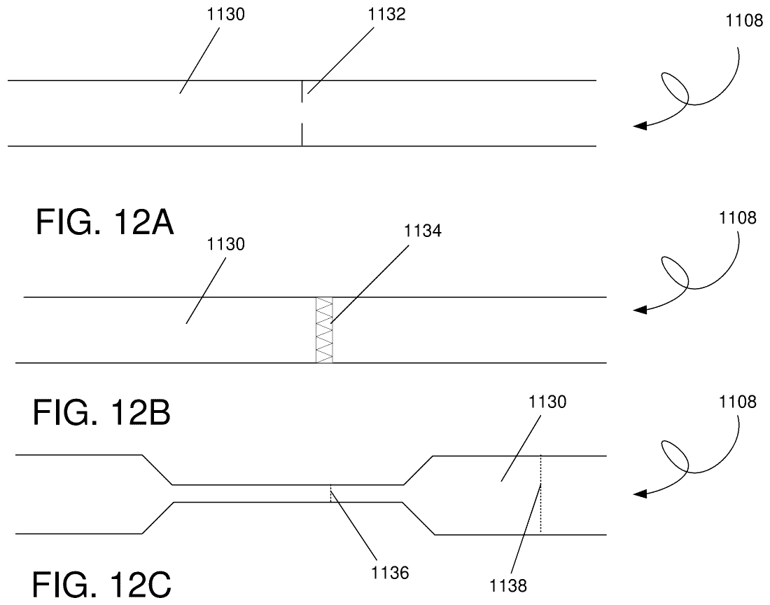

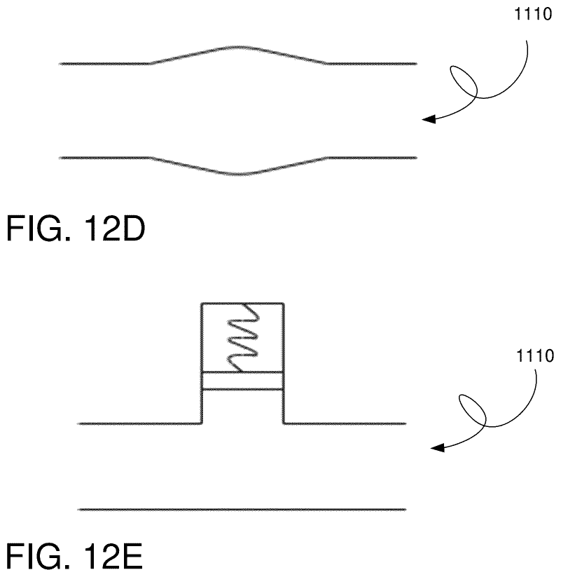

FIGS. 12A, 12B, and 12C are schematics of the resistive component 1108 comprising tubing 1130 with fixed orifice 1132, tube with inline filter 1134, and tube with ID 1136 smaller than ID 1138. As shown in FIG. 12A, the filter 1134 may comprise an orifice 1132. As shown in FIG. 12B, orifice 1132 may be configured in the tubing 1130 connecting to the reference port 1120. In further embodiments, the orifice may be adjustable. As shown in FIG. 12B, the tubing 1130 connecting to the reference port 1120 may be configured with an in-line filter 1134. As shown in FIG. 12C, the tubing 1130 connecting to the reference port 1120 may include a portion with a reduced diameter, or a small ID 1136 relative to the ID of the tubing 1138.

In some implementations, the filter 1134 may comprise a fixed or adjustable orifice 1132. The orifice 1132 may control the amount of air that can flow between the rest of the BP pressure cuff system and the reference port 1120. Air flow across the orifice 1132 is driven by a pressure differential. Limiting flow between compartments using an orifice instead of a valve results in smoothing out pressure oscillations while maintaining mean signal, acting as low pass filter on reference port side.

FIGS. 12D and 12E are schematics of the capacitive component 1110 including an elastic tube (FIG. 12D), and tube with a piston cylinder (FIG. 12E).

An important measurement may be the difference between the signal measured at the measurement port and at the reference port. Therefore, the output signal is the equivalent of a high pass filtered signal. Additionally, a self-adjusting reference port signal may also maintain a centered output signal. Using an orifice, as opposed to an air valve, allows the reference port to stay at the mean pressure signal. Maintaining the mean pressure signal is important to eliminate bias in the measured pulsations and maintain a centered signal with the high-resolution transducer.

In some implementations, the hydrodynamic filter may comprise a resistive component comprising a tube with a small ID. In such embodiments, the resistive component may comprise substantially rigid tubing with an internal diameter in the range of 10-200 μm. The ID of the tube may be significantly smaller in diameter than the ID of the tubing connected the rest of the BP pressure cuff system and reference port. The tubing with small ID effectively acts as a filter in which only the mean pressure is transmitted creating a flow dependent low pass filter to the reference port. Specific cutoff frequencies of the filter are designed using fluid dynamic principles and the characteristics of the measuring system and signal. The filtered signal will be dependent on the combination of volumetric flow rate and the material properties of the reference port side.

In some implementations, the system may be designed using a commercial arm cuff BP system. The system may be modified to include a plurality of pressure sensors with different operating ranges to measure and calibrate the pressure waveform with high accuracy. High resolution pressure sensors may be used for accurate signal measurement. Each high resolution pressure sensor may contain a measurement port and a reference port. High range pressure sensors may be used for absolute reference and signal calibration.

In some implementations, the system can be applied to any location in the body that has arteries close to the surface and can withstand a brief reduction or cessation of blood flow. Potential locations may include, but are not limited to, brachial, radial, femoral, and posterior tibial.

A pressure sensor may measure peripheral pressure pulse. Per fluid-solid interaction principles, the pressure at which cuff is inflated alters pressure-flow behavior in artery. Combinatorial waveform analysis may then be used to non-invasively assess cardiovascular health. A peripheral pulse waveform may be measured and then may be transformed to estimate the central waveform.

Pressure and flow velocity in closed system may be given by the Bernoulli equation (below). When a brachial cuff is inflated, the externally applied force alters the radius of the artery, ultimately changing the pressure flow proportion of the system. At a lower extreme, applying pressure to the artery below minimum DBP will cause no or minimal alteration to pressure flow behavior. At an upper extreme, pressure above the maximum SBP in the artery causes collapse of artery and cessation of blood flow. Any pressure between these two extremes may create a proportional alteration to the pressure flow relationship, again given by the Bernoulli equation. Comparing a measured waveform at two different hold pressures therefore allows derivation of the pressure-flow characteristics of system. Quantitative and qualitative comparisons of the waveforms may be performed. Plotting methods may also be used such as waveform versus time or waveform versus waveform.

Captured signals reflecting the pressure flow relationship in elastic arteries can be used to derive additional waveforms that may further characterize a patient. Fluid dynamic principles enable deriving waveforms, including, but not limited to, flow, velocity, and radial movement. Fluid-dynamics principles relate parameters such as pressure, velocity, forces, and volumes for static systems. Analyzing these systems with multiple measurement points allows to solve for the interrogated parameters. For example, velocity may be solved for by using DBP and sSBP hold pressure waveforms. The sSBP waveform completely obstructs flow, giving an absolute pressure reading. The DBP waveform represents a combination of pressure and flow. Therefore, the resulting flow may be measured during the DBP hold pressure. Similar derivations may be applied to other hold pressure combinations. The significance of the results obtained may depend on the underlying physics of the captured waveform(s).

Practical applications may require synchronization of waveforms. Solutions may include time synchronization of different waveforms using ECG or using known timing events during cardiac cycle including max dP/dt, start, and dicrotic notch. This cuff can have wired or wireless synchronization with other devices, such as Bluetooth or Wi-Fi with ECG.

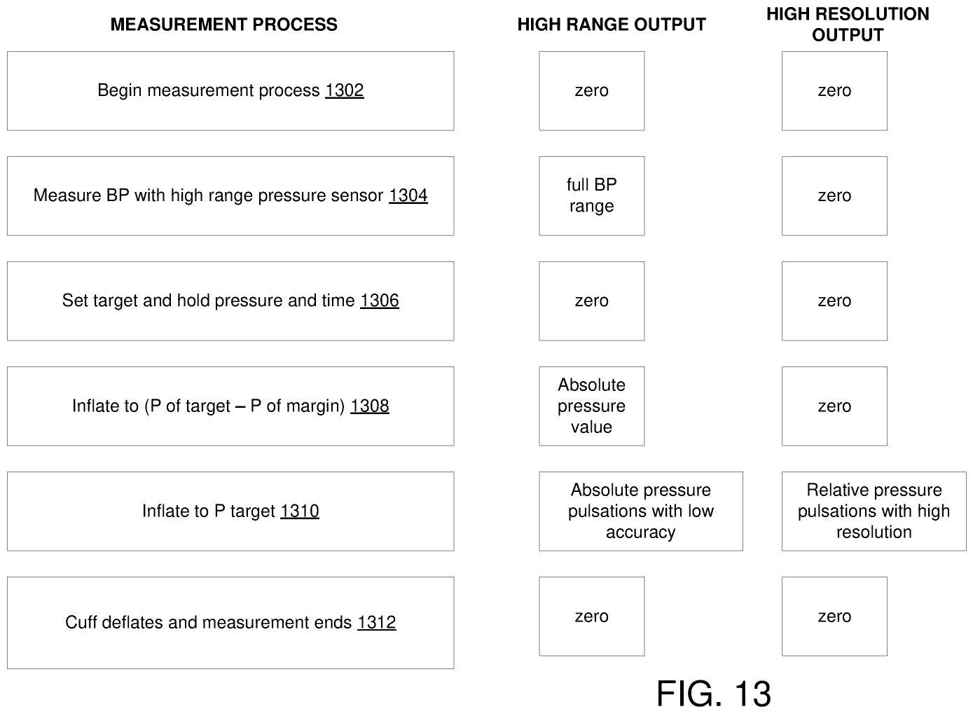

FIG. 13 shows an example of a non-invasive pulse pressure waveform measurement. A modified BP pressure cuff assembly, as described above, may be used to record pressure pulsations in the brachial artery. To perform the measurement a brachial BP cuff may be inflated around a patient's arm. A pump system may be used to inflate the cuff. A first operation 1302 may involve starting the measurement process. At the time of the first operation 1302, the output of a high range pressure sensor and a High resolution pressure sensor may both be zero. The pressure sensor may be used to measure the SBP and DBP of the patient. A second operation 1304 may involve measuring BP with a high range pressure sensor. At the time of the second operation 1304, the output of a high range sensor may the full BP range for the patient and the output of a high resolution pressure sensor may be zero.

As a third operation 1306, target and hold pressure and time may be set. For example, the cuff may be set to be inflated to a pressure of 100 mmHg and may be held for 40 seconds. Other pressures and timing are also possible. At the time of the third operation 1306, the output for both the high range and high resolution pressure sensors may be zero. Inflation pressure references and/or targets for the cuff may be obtained by performing traditional blood pressure arm cuff measurements. For example, hold pressures may be set at DBP, below DBP, at SBP, above SBP, and/or at MAP. Typical physiological ranges for these values are as follows: 40-120 mmHg for DBP; 50-150 mmHg for MAP; and 75-225 mmHg for SBP. Other pressures are also possible. For example, extremely sick subjects could have values outside of these ranges. For patient specific values, a specified pressure level may be employed to guide the hold pressure selection. To maintain the high resolution pressure sensor within its operating range, a pressure may applied to the reference port.

As a fourth operation 1308, the cuff may be inflated and held at a given pressure. For example, the cuff may be inflated to a pressure of P target, which may be one of the identified hold pressures. At the time of the fourth operation 1308, the high range sensor output may be the absolute pressure value and the high resolution pressure output may be zero. As a fifth operation 1310, the cuff may then be inflated to the target pressure. At the time of the fifth operation 1310, the high range sensor may have an output of the absolute pressure pulsations with low accuracy. The high resolution sensor may have an output of the relative pressure pulsations with high accuracy. As a sixth operation 1312, the cuff may deflate, ending the measurement period. At the time of the sixth operation 1312, each of the high and high resolution pressures sensors may have an output of zero. For multiple hold pressures, operations three to six may be repeated. Outputs from operation five may be combined for a calibrated high accuracy pulsation output.

In some implementations, comparisons of waveform captures may be performed using a low hold pressure and a high hold pressure above an upper pressure extreme. For instance, for the low hold pressure, pressure may be set at or just below DBP. For the high hold pressure, pressure may be at sSBP, cutting off the flow of blood. For example, pressure may be set at about SBP+35 mmHg. At the lower extreme, the waveform may represent a combination of static pressure and flow velocity. At the upper extreme, waveform only displays pressure characteristics. At sSBP hold pressures, cessation of flow in subclavian artery becomes closest waveform for representing static pressure read from a hole in the wall of the ascending aorta. This hold pressure allows for direct pressure waveform measurements in central arteries. Respective pressure waveform between sSBP and DBP can be plotted in pressure-pressure (“PP”) loop for cardiac health and disease assessment. This allows for creation of pressure-velocity (“PV”) loop applied for health assessments.

### Pressure-Velocity (“PV”) Loop Embodiment

As discussed above, flow at the brachial artery may be characterized with the Bernoulli equation for average flow, given by:

$${P}_{B} + \frac{1}{2} ρ {u}_{B}^{2} = {P}_{T}$$

where P_{B} is the pressure at the brachial artery, ρ is the fluid density, u_{b} is the flow velocity at the brachial artery, and P_{T} is the total pressure in the aortic arch.

At the sSBP pressure hold (P_{SS}), the brachial artery is completely occluded resulting in the u_{b}=0. Thus, the measured pressure at the cuff is the pressure in the aortic arch. As such:

P_{SS}=P_{B}=P_{T}

where P_{SS} is the sSBP hold pressure.

At the DBP pressure hold (P_{D}), the applanation condition measures the pressure in the brachial artery. The pressure in the brachial artery fits in the Bernoulli equation as shown below:

$${P}_{D} = {P}_{B} \to {P}_{B} + \frac{1}{2} ρ {u}_{B}^{2} = {P}_{T}$$

where P_{D} is the DBP hold pressure.

Equating the above through the total aortic arch pressure and solving for the velocity (u_{B}) gives:

$${P}_{S S} = {P}_{D} + \frac{1}{2} ρ {u}_{B}^{2}$$

$${u}_{B} = \sqrt{\frac{2}{ρ} * ( {P}_{S S} - {P}_{D} )}$$

Plotting the sSBP pressure (P_{SS}) versus the DBP velocity (u_{D}) gives the (“PV”) loop. Data may be plotted and analyzed at any intermediate step. For example, SBP pressure versus DBP pressure may be analyzed.

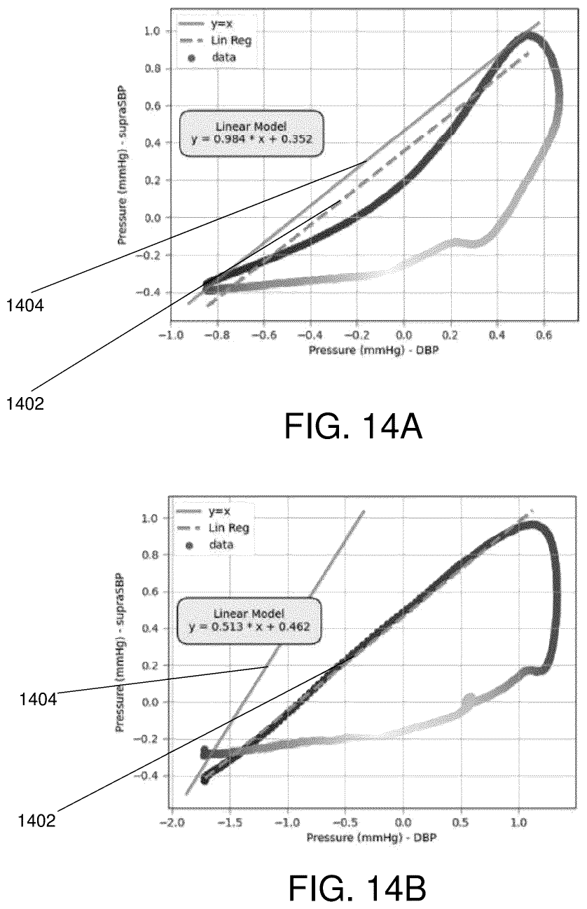

FIGS. 14A-14B shows examples of PV loop comparison of older (FIG. 14A) and younger (FIG. 14B) patients. Each of the PV loops have different features and shapes. Each includes a dashed line 1402 which models the slope of rising systolic portion. The slope can clearly differentiate patients by age, so it is a useful diagnostic. Each also includes a solid line 1404 which is a proportionality line. Other parameters include loop areas, curvatures, indentations, peak shifts, and other combinations of parameters.

### Left Ventricular End-Diastolic Pressure (“LVEDP”) Risk Prediction Embodiment

LVEDP is an important clinical measurement used to predict, diagnose, and/or assess the risk for heart failure. Currently, the threshold for heart failure is an LVEDP measurement of about 18 mmHg. Because LVEDP is a valuable diagnostic measurement, non-invasive methods for measuring LVEDP may allow clinicians to predict risk of heart failure earlier and with accuracy.

A non-invasive pulse waveform analysis and classification algorithm may be used to form an LVEDP risk prediction.

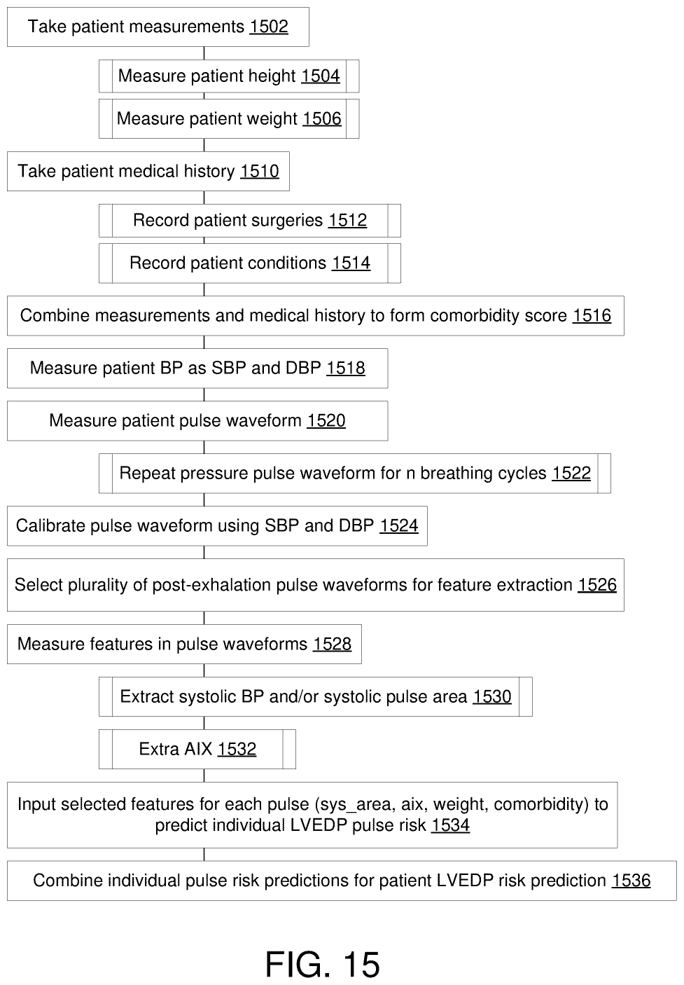

FIG. 15 shows an example of a non-invasive LVEDP risk prediction method 1500 involving several operations. A first operation 1502 may involve taking patient measurements. A sub operation 1504 of the first operation 1502 may involve measuring the patient's height. A sub operation 1506 of the first operation 1502 may involve measuring the patient's weight. Other patient measurements may also be taken. A second operation 1508 may involve taking a patient's medical history. A sub operation 1510 to the second operation 1508 may involve recording patient surgeries. Patient surgeries, such as heart surgeries, may be recorded. Other medical information, such as procedures and other treatments may also be recorded. A sub operation 1512 to the second operation 1508 may involve recording patient conditions. Patient conditions may include known instances of cardiovascular conditions such as heart failure, myocardial infarction, and cardiomyopathy. Other heart conditions, comorbid conditions, and/or general health conditions may be recorded. Other patient information may also be recorded such as genetic predispositions, family history, lifestyle factors and other information.

A third operation 1516 may involve combining recorded patient measurements and medical history to form a comorbidity score.

A fourth operation 1518 may involve measuring the patient's BP as SBP and DBP using a commercially available and/or conventional brachial cuff or some other measurement means.

A fifth operation 1520 may involve measuring the patient's pulse waveforms. The pulse waveforms may be measured using a modified BP cuff in accordance with the foregoing embodiments. For example, a modified blood pressure cuff that inflates to specific pressures and holds those pressures to capture a waveform at the set pressure may be used. Hold pressures may include DBP, SBP, MAP, and/or sSBP. In some embodiments, an sSBP, which completely cuts off the flow of blood through an artery, may be used. The inflation pressure may be, for example, about 100 mmHg. The hold time may be about, for example, 40 seconds.

Sub operation 1522 to the fifth operation 1520 may involve performing the pulse waveform measurements for a duration long enough to account for pressure amplitude fluctuations throughout a breathing cycle. Measured amplitudes may be highest at the post-exhalation stage of the breathing cycle.

A sixth operation 1524 may include calibrating the measured pulse waveform(s) using the SBP and DBP measurements. The waveform may be calibrated with pressure units utilizing BP measurement results. Calibration methods may include the methods disclosed in the following section of this disclosure.

A seventh operation 1526 may include selecting a plurality of post-exhalation waveforms for feature extraction. Post-exhalation waveforms may be selected because these waveforms may track the highest LVEDP reading throughout a breathing cycle. Extracted features and/or parameters of interest may include augmentation index (“AIX”), systolic pulse area, and/or systolic BP. Other desirable and/or relevant features and/or parameters may also be extracted.

An eighth operation 1528 may include measuring and/or extracting the features and/or parameters of interest in the pulse waveforms. A sub operation 1530 to the eight operation 1528 may involve extracting SBP or systolic pulse area. A sub operation 1532 to the eighth operation 1528 may involve extracting the AIX.

In some embodiments, a classification algorithm may be used to assess risk. Inputs for the algorithm may be pulse features of systolic pulse area, augmentation index and patient features of weight and comorbidity score. Using the inputs and algorithm, a probability of having LVEDP greater than or equal to a failure threshold may be generated. In an embodiment, the failure threshold may be set at about 18 mmHg. In another embodiment, the threshold may be set at about 15 mmHg or at another value of clinical relevance selected when training the algorithm. This process may be repeated for post-exhalation pulses in n breathing cycles to generate n probability predictions. A plurality of measurements may be taken. For example, in an embodiment, two or three measurement may be taken. The probability of the plurality of individual pulses may be combined into a single risk prediction using ensemble methodologies. The predictions may be processed together to generate a single LVEDP risk prediction. Ensemble methodologies may include averaging the probabilities and/or may include more complex methods of aggregating the probabilities.

Accordingly, a ninth operation 1534 may involve inputting selected features and/or parameters for each pulse to predict individual LVEDP risk. The selected features and/or parameters may include the systolic pulse area, AIX, patient weight, and comorbidity score. Other parameters and/or combinations of parameters are also possible. Likewise, a tenth operation 1536 may involve combining individual pulse risk predictions for patient LVEDP risk prediction.

### Calibration Embodiment

BP cuff measurements may serve as useful clinical measurements because peripheral BP, as measured via a cuff, tends to track central BP in healthy patients. Unfortunately, in patients experiencing cardiovascular issues, the relationship between peripheral BP and central BP may degrade. The severity of the cardiovascular issues in a patient may affect the extent to which the peripheral-central BP relationship degrades. However, because BP cuff measurements are quick, non-invasive, and inexpensive to perform, BP cuff measurements remain an important diagnostic tool for patients experiencing cardiovascular issues. Though the peripheral-central BP relationship degrades in such patients, calibration methods may allow a peripheral BP measurement performed with, for example, a brachial BP cuff, to serve as a proxy for a central BP measurement even in a patient experiencing severe issues.

A modified BP cuff system, such as systems described in the foregoing embodiments, may be used to measure peripheral BP. Peripheral BP may be measured over several breathing holds cycles due to pressure fluctuations caused by breathing. A non-invasive pulse signal measured using a modified BP cuff system may be calibrated to track central BP magnitudes in a patient.

An envelope function may be used to correct a peripheral BP measurement and calibrate a signal. The envelope function may comprise a relationship between pulse amplitude and cuff pressure at measurement site. The measurement cite may be an artery. An envelope function may be constructed by measuring pulse amplitude corresponding to cuff pressure across multiple breath holds.

A calibration method may include several operations. A first operation may involve measuring peripheral BP in the form of SBP, DBP, and MAP using a conventional and/or commercially available oscillometric cuff. These measured values may not be accurate. Specifically, these measured values may not track measurement taken in vivo due to amplitude fluctuations caused by breathing and/or due to other errors.

A second operation may involve using a modified BP cuff. The modified BP cuff may be a modified BP system including high resolution pressure sensors and high range pressure sensors, as described in the foregoing embodiments. The second operation may involve inflating the modified BP cuff to a set pressure value. Pulsations may be recorded at that pressure value. The BP cuff may be inflated over a range of set pressure values. Pulsations may be recorded for each pressure value. The second operation may involve taking operations over multiple breath holds to account for pressure fluctuations caused by breathing. For each breath hold, the measured waveform may be analyzed to compare signal pulse amplitude to the set pressure of the modified BP cuff. Measurements may be taken over a plurality of holds to reconstruct a proxy of the envelope function. For example, measurements may be taken over two, three, or more breath holds.

A third operation may involve using the measurements taken during the second operation to calculate parameters to correct for pressure fluctuations due to breathing changes and ultimately to calibrate peripheral measurements to central measurements. This may be accomplished by using an envelope function to derive the parameters needed to correct the peripheral measurements.

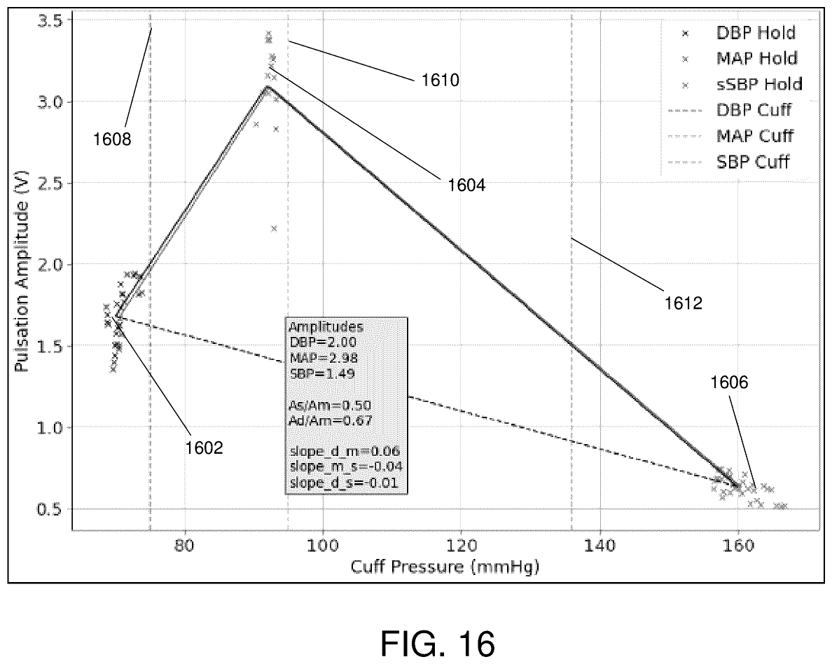

FIG. 16 shows an example of an envelope function reconstructed with a BP measurement and three pressure holds. The three pressure holds are DBP, MAP, and sSBP. For the DBP pressure hold, the modified BP cuff was inflated with minimal pressure, allowing blood to flow through the artery substantially unobstructed. For the MAP pressure hold, the modified BP cuff was inflated to MAP. For the sSBP hold, the modified pressure cuff was inflated above the pressure for SBP to effectively cut off any blood flow through the artery. Cuff pressure is shown in FIG. 16 as dashed vertical lines for the three hold values. The three holds values include the DBP hold 1608, the MAP hold 1610, and the sSBP hold 1612.

FIG. 16 also shows individual record pulsations measured at each hold pressure. FIG. 16 shows individual pulsations 1602 for the DBP hold, individual pulsations 1604 for the MAP hold, and individual pulsations 1606 for the sSBP hold. As shown in the example of FIG. 16, multiple pulsations may be recorded for each pressure hold. The pulse amplitudes of the individual pulsations 1602, 1604, 1606 may be measured with a pressure sensor. In the example shown in FIG. 16, pulsation amplitude is reported in volts (V). These pulse amplitude measurements may also be converted into other pressure units.

As discussed above, breathing may cause substantial fluctuations in central BP. The voltage-based signals from a cuff pressure hold may show breathing fluctuations just as in a catheter aortic signal. Therefore, BP values reported by a modified BP cuff measurement may be assumed to be mean values. Pulse signals may be calibrated to pressure units by adjusting SBP and DBP values over a breath pattern and correctly scaling the measured pressure signal. For each individual pulsation in a segment of pulsations, the pulse amplitude difference from the mean pulse amplitude of the segment may be used to correct the SBP and/or DBP values for breathing fluctuations.

For example, a model to correct DBP values from peripheral to central DBP using the envelope function derived parameters is given by:

$$D B {P}_{corr} = D B {P}_{c u f f} + {m}_{1} * \frac{{A}_{d}}{{A}_{m}} + {m}_{2} * M A {P}_{c u f f} + b$$

where A_{d}/Aₘ is the ratio between the pulse amplitude at DBP versus MAP and DBP_{cuff} and MAP_{cuff} are the DBP and MAP reported by the cuff BP reading respectively, and m₁, m₂, and b are coefficients optimized for the correlation.

SBP values may be corrected using forward and reflect wave peaks measurable in a pulse waveform signal. For the brachial cuff, a potential hold pressure that shows these features is sSBP. Corrected SBP may be given by:

SBP_{corr}=SBP_{cuff}+m₁*(P₁−P₂)+b

where SBP_{corr} is the corrected SBP value to track central BP, SBP_{cuff} is the SBP cuff measurement readout, P₁ is the peak pressure of the first peak in systole, P₂ is the peak pressure of the second peak in systole, and m₁ and b are coefficients optimized for the correlation.

A linear envelope function model may be used to calculate the actual pressure as shown in the following close form equation:

$${P}_{a d j} = {P}_{c a l i b} + \frac{Δ P A}{{slope}_{p}}$$

where P_{adj} is the breathing adjusted pressure, P_{calib} is the BP reported value, ΔPA is the pulse amplitude difference from mean, and slopeₚ is the envelope function slope for the specific pressure (slope_{DBP} or slope_{SBP}). Pressure may be either SBP or DBP.

In an uncalibrated segment of pulsations, repeating the foregoing calculations for every pulsation in the segment of pulsations and utilizing signal scaling methodologies, all pulsations can be calibrated with SBP and DBP values that reflect breathing patterns. The model presented assumes a linear relationship between measurement points and a fixed envelope function for a given subject. With more hold pressures, a more detailed envelope function may be reconstructed and may increase model accuracy. Calibration may be applied to SBP and/or DBP independently.

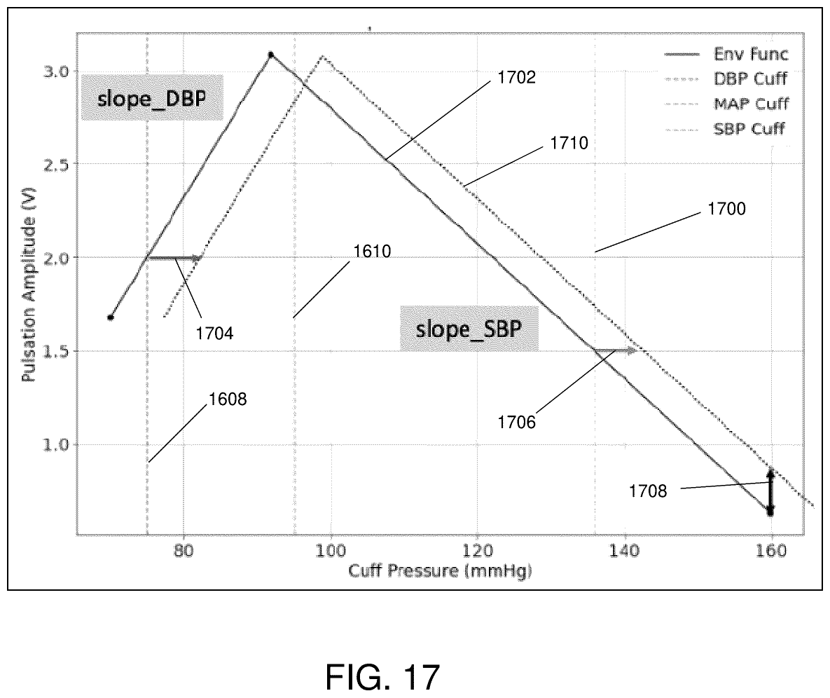

FIG. 17 shows an example of an envelope function used to estimate SBP and DBP changes following pulse amplitude fluctuations. As in FIG. 16, pressure cuff holds are represented by dashed vertical lines. FIG. 17 shows a DBP pressure cuff hold value 1608, a MAP pressure cuff hold value 1610, and a SBP pressure cuff hold value 1700. The arrow 1708 at the bottom right of FIG. 17 shows the pulse amplitude deviation from mean pulse amplitude. The arrow 1706 above and right of the arrow 1708 shows the SBP increase. The arrow 1704 at the left of FIG. 17 shows the DBP increase. The envelope function 1702 can then be used to estimate SBP and DBP changes following fluctuations in pulse amplitude, which may be caused by breathing. FIG. 17 shows the estimate 1710. FIG. 17 is exemplary only. The methodology described above with reference to FIG. 17 may be performed with different devices, threshold conditions, and quantities if the necessary information described is obtained.

In some embodiments, a calibration method incorporating breathing fluctuations may also serve as a diagnostic tool in cardiology. For example, the condition of pulsus paradoxus is defined as a fall in SBP greater than about 10 mmHg during inspiration. This condition may be observed during cardiac tamponade or right ventricle distension such as in severe acute asthma or chronic obstructive pulmonary disease. Therefore, an embodiment of a calibration method may involve setting a threshold of about 10 mmHg.

In this document, the terms “machine readable medium,” “computer readable medium,” and similar terms are used to generally refer to non-transitory mediums, volatile or non-volatile, that store data and/or instructions that cause a machine to operate in a specific fashion. Common forms of machine readable media include, for example, a hard disk, solid state drive, magnetic tape, or any other magnetic data storage medium, an optical disc or any other optical data storage medium, any physical medium with patterns of holes, a RAM, a PROM, EPROM, a FLASH-EPROM, NVRAM, any other memory chip or cartridge, and networked versions of the same.

These and other various forms of computer readable media may be involved in carrying one or more sequences of one or more instructions to a processing device for execution. Such instructions embodied on the medium, are generally referred to as “instructions” or “code.” Instructions may be grouped in the form of computer programs or other groupings. When executed, such instructions may enable a processing device to perform features or functions of the present application as discussed herein.

In this document, a “processing device” may be implemented as a single processor that performs processing operations or a combination of specialized and/or general-purpose processors that perform processing operations. A processing device may include a CPU, GPU, APU, DSP, FPGA, ASIC, SOC, and/or other processing circuitry.

The terms “substantially” and “about” used throughout this disclosure, including the claims, are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

To the extent applicable, the terms “first,” “second,” “third,” etc. herein are merely employed to show the respective objects described by these terms as separate entities and are not meant to connote a sense of chronological order, unless stated explicitly otherwise herein.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

The terms “substantially” and “about” used throughout this disclosure, including the claims, are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

To the extent applicable, the terms “first,” “second,” “third,” etc. herein are merely employed to show the respective objects described by these terms as separate entities and are not meant to connote a sense of chronological order, unless stated explicitly otherwise herein.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present disclosure. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described exemplary embodiments.

It should be appreciated that all combinations of the foregoing concepts (provided such concepts are not mutually inconsistent) are contemplated as being part of the inventive subject matter disclosed herein. In particular, all combinations of claimed subject matter appearing in this disclosure are contemplated as being part of the inventive subject matter disclosed herein.

What is claimed is:

1. A method for noninvasively performing cardiac auscultation, comprising:
performing, using a blood pressure (BP) cuff of a BP cuff system, a blood pressure measurement of a subject to obtain one or more blood pressure values corresponding to the subject;
inflating, based on the one or more blood pressure values corresponding to the subject, the BP cuff to a subject specific pressure value;
capturing, using the BP cuff system, while the BP cuff is inflated to the subject specific pressure value, a pulse pressure waveform signal associated with an artery of the subject;
obtaining a sound waveform signal associated with a heart valve of the subject by filtering the pulse pressure waveform signal to extract sound components associated with an opening or closing of the heart valve; and
analyzing the sound waveform signal to identify characteristics of the heart of the subject.

2. The method of claim 1, wherein:
the one or more blood pressure values comprise a systolic blood pressure (SBP) of the subject; and
the subject specific pressure value comprises a supra systolic blood pressure (sSBP) greater than the SBP.

3. The method of claim 2, wherein the BP cuff is a brachial cuff, the artery is a brachial artery of the subject, and the heart valve is an aortic valve of the subject.

4. The method of claim 1, further comprising: prior to analyzing the sound waveform signal, indexing the sound waveform signal to identify cardiac cycle events of the subject.

5. The method of claim 4, wherein indexing the sound waveform signal comprises indexing, based on the pulse pressure waveform signal, the sound waveform signal to identify the cardiac cycle events.

6. The method of claim 4, wherein:
the method further comprises: capturing, concurrently to the BP cuff system capturing the pulse pressure waveform signal, an electrocardiogram (ECG) of the subject; and
indexing the sound waveform signal comprises indexing, based on the ECG, the sound waveform signal to identify the cardiac cycle events.

7. The method of claim 1, wherein filtering the pulse pressure waveform signal to extract sound components associated with the opening or closing of the heart valve comprises filtering the pulse pressure waveform signal to exclude signal components having a frequency below about 18 Hz.

8. The method of claim 7, wherein filtering the pulse pressure waveform signal to extract sound components associated with the opening or closing of the heart valve further comprises filtering the pulse pressure waveform signal to exclude signal components having a frequency above about 250 Hz.

9. The method of claim 1, wherein analyzing the sound waveform signal to identify characteristics of the heart comprises:
analyzing a first component of the sound waveform signal associated with opening of the heart valve; or
analyzing a second component of the sound waveform signal associated with closing of the heart valve.

10. The method of claim 9 wherein analyzing the sound waveform signal to identify characteristics of the heart comprises measuring a stiffness of the heart valve based on an amplitude of the first component or the second component of the sound waveform signal during one or more cardiac cycles.

11. The method of claim 9 wherein analyzing the sound waveform signal to identify characteristics of the heart comprises measuring a stiffness of the heart valve based on a presence or absence of the first component or second component of the sound waveform signal during one or more cardiac cycles.

12. The method of claim 9 wherein analyzing the sound waveform signal to identify characteristics of the heart comprises measuring a contraction strength of the heart based on one or more parameters of the first component of the sound waveform signal associated with opening of the heart valve.

13. The method of claim 9 wherein analyzing the sound waveform signal to identify characteristics of the heart comprises measuring a relaxation strength of the heart based on one or more parameters of the second component of the sound waveform signal associated with closing of the heart valve.

14. The method of claim 1, wherein analyzing the sound waveform signal to identify characteristics of the heart comprises: generating, based on one or more features of the sound waveform signal, using a trained machine learning model, a prediction output indicating whether or not the sound waveform signal is associated with a heart condition.

15. A non-transitory computer-readable medium having executable instructions stored thereon that, when executed by a processor, cause the processor to perform operations comprising:
obtaining a pulse pressure waveform signal associated with an artery of a subject;
obtaining a sound waveform signal associated with a heart valve of the subject by filtering the pulse pressure waveform signal to extract sound components associated with an opening or closing of the heart valve;
indexing the sound waveform signal to identify cardiac cycle events of the subject; and
analyzing the indexed sound waveform signal to identify characteristics of the heart of the subject.

16. The non-transitory computer-readable medium of claim 15, wherein filtering the pulse pressure waveform signal to extract sound components associated with the opening or closing of the heart valve comprises filtering the pulse pressure waveform signal to exclude signal components having a frequency below about 18 Hz.

17. The non-transitory computer-readable medium of claim 16, wherein filtering the pulse pressure waveform signal to extract sound components associated with the opening or closing of the heart valve further comprises filtering the pulse pressure waveform signal to exclude signal components having a frequency above about 250 Hz.

18. The non-transitory computer-readable medium of claim 16, wherein analyzing the indexed sound waveform for potential heart conditions comprises:
analyzing a first component of the sound waveform signal associated with opening of the heart valve; or
analyzing a second component of the sound waveform signal associated with closing of the heart valve.

19. The non-transitory computer-readable medium of claim 18, wherein analyzing the indexed sound waveform signal to identify characteristics of the heart comprises measuring a stiffness of the heart valve, a contraction strength of the heart, or a relaxation strength of the heart based on one or more features of the first component of the sound waveform or the second component of the sound waveform.

20. The non-transitory computer-readable medium of claim 15, wherein analyzing the indexed sound waveform signal to identify characteristics of the heart comprises: generating, based on one or more features of the sound waveform signal, using a trained machine learning model, a prediction output indicating whether or not the sound waveform signal is associated with a heart condition.
